(19) United States
(12) Patent Application Publication
(10) Pub. No.: US 2010/0130544 A1
(43) Pub. Date: May 27, 2010

(54) USE OF PARASYMPATHOLYTIC SUBSTANCES TO ENHANCE AND ACCELERATE STEM CELL DIFFERENTIATION, RELATED METHODS AND COMPOSITIONS

(75) Inventors: Antonio Ponzetto, Moncalieri (Torino) (IT); Luisa Gennero, Torino (IT); Maria Augusta Roos, Torino (IT); Gian piero Pescarmona, Torino (IT); Tetyana Denysenko, Torino (IT); Paolo Di Nardo, Rome (IT); Roberto Sangiorgio, Monza (Milano) (IT)

Correspondence Address: Steinfl & Bruno, 301 N Lake Ave Ste 810, Pasadena, CA, 91101, US

(73) Assignee: UNIVERSITA' DEGLI STUDI DI TORINO, Torino (IT)

(21) Appl. No.: 12/301,392

(22) PCT Filed: Feb. 28, 2007
(86) PCT No.: PCT/IB2007/050644

§ 371 Date: Nov. 18, 2008

(30) Foreign Application Priority Data
May 19, 2006 (IT) TO2006A000365

(51) Int. Cl. A61K 31/4748; C12N 5/071; A61K 31/44; A61K 31/22; A61P 35/00

(52) U.S. Cl. 514/304; 435/377; 435/404; 435/405; 435/406; 514/357; 514/546

(57) ABSTRACT

The invention refers to the in vitro and in vivo use of parasympatholytic substances, preferably scopolamine, to potentiate and accelerate the differentiation of stem cells into cells with a tissue-specific phenotype, and the process and compositions related thereto.

The present invention concerns a novel use for parasympatholytic substances and, more particularly, the use of parasympatholytic substances to enhance and accelerate the differentiation of stem cells, preferably pluripotent stem cells, into cells with tissue-specific phenotypes, the method and the compositions related thereto.

It has long been known that stem cells can be maintained indefinitely in vitro in an undifferentiated state, i.e. allowed to proliferate by increasing in number, or also be “guided” to differentiate into hepatic, myocardial, neural, pancreatic cells etc., by using different inducing culture media, principally based on growth factors or other substances capable of inducing the differentiation of the stem cells into a tissue-specific phenotype.

The present invention aims at providing substances capable of stimulating, enhancing and accelerating the differentiation and proliferation of stem cells into cells with a tissue-specific phenotype.

According to the present invention, said aim is achieved by means of the solution specifically claimed in the following claims. The claims form an integral part of the technical teaching provided herein in relation to the invention.

The invention relates to the use of parasympatholytic substances for stimulating, enhancing and accelerating the differentiation and proliferation of stem cells into cells with a tissue-specific phenotype.

Said ability of the parasympatholytic substances to stimulate, enhance and accelerate the differentiation and proliferation of stem cells into cells with a tissue-specific phenotype, may be exploited in both in vitro and in vivo applications.

Hence, according to one embodiment, the invention concerns the use of at least one parasympatholytic substance for preparing a medicament for stimulating, enhancing and accelerating the differentiation and proliferation of stem cells into cells having a tissue-specific phenotype in a subject in need thereof.

In another embodiment, the present invention concerns a culture medium supplemented with at least one parasympatholytic substance capable of stimulating, enhancing and accelerating the differentiation and proliferation of stem cells into cells with a tissue-specific phenotype.

The culture medium of the invention is preferably a culture medium adapted for the growth of eukaryotic cells, preferably mammalian cells and even more preferably human cells, namely a solid or liquid solution comprising the usual salts, amino acids, sugars, peptides, vitamins and/or vitamin factors required for the growth of the above-specified cells.

The present invention further relates to a method for stimulating, enhancing and accelerating the differentiation and proliferation of stem cells into cells having a tissue-specific phenotype, wherein said stem cells are grown in the presence of at least one parasympatholytic substance and one tissue-specific inducing growth factor.

Purely by way of non-limiting example, the invention will now be described in detail with reference to some particularly preferred embodiments.

The observed in vivo proliferative stimulatory effects of certain sympathomimetic substances are well known.

The present invention is based on the in vitro observation of the ability of parasympatholytic substances to stimulate the cellular differentiation of stem cells. Such substances, when added to a phenotype-inducing culture medium, induce the stem cells in question to undergo accelerated differentiation and subsequent cell differentiation. Hence, in the present invention, parasympatholytic substances have been used for the first time as activators of growth factors (for example, HGF, hepatocyte growth factor; MCSF, macrophage colony stimulating factor) and as inducers of the accelerated differentiation and proliferation of stem cells into a tissue-specific phenotype. The acceleration, with respect to the inducing culture media previously studied by the present inventors (see for example the Italian patent applications Nos. TO2005A000800 and TO2005A000819) is demonstrated by the reduced time required to achieve differentiation: 7-10 days, instead of 15-28 days.

The compositions (i.e., culture media and medicaments) forming the subject of the present invention offer the following advantages:
excellent growth and recovery of physiological trophism when the compositions are added to standard stem cell-specific culture media;
rapid in vitro differentiation of stem cells into a tissue-specific phenotype, when a tissue-specific-inducing culture medium is used;
all the tissue-specific-phenotypes obtained have been frozen at −80° C., thawed, and reactivated in culture for a further month, in order to ascertain consistent normal histo-functionality.

Materials and Methods

The compositions forming the subject of the present invention have been prepared using, interalia, the substances reported below:

1. Amino Acids

Methionine, cystine, N-acetylcysteine, cysteine, glycine, leucine, isoleucine, proline, glutamine, arginine, glutamic acid, histidine, histidine-HCl—H₂O, lysine, lysine-HCl, phenylalanine, serine, threonine, tryptophane, tyrosine, tyrosine-disodium salt, valine, proline, hydroxyproline, a solution containing all the non-essential amino acids.

2. Peptides

Glutathione, collagen, elastin, wheat extract, polypeptides to which are attributed trophic functions.

3. Vitamins

Retinoic acid, retinol, ascorbic acid, pantothenic acid, D-calcium pantothenate, pyridoxine, pyridoxine-HCl, folic acid, niacinamide, riboflavin, cobalamine, para-aminobenzoic acid and biotin.

4. Vitamin Factors

Inositol, myo-inositol, choline chloride, pyruvic acid, sodium pyruvate, putrescine and putrescine-HCl.

5. Growth Factors

TGF-Beta (transforming growth factor beta), LIF (leukaemia inhibitory factor), ITS (insulin-transferrin-selenium), insulin, HGF (hepatocyte growth factor), M-CSF (macrophage colony stimulating factor), IL-2 (Interleukin-2), IL-6 (Interleukin-6), PMA (phorbol-12-myristate-13-acetate), linoleic acid, autologous serum.

6. Salts

Calcium gluconate, disodium dexamethasone 21-phosphate, calcium phosphate, sodium bicarbonate, calcium chloride, magnesium chloride, magnesium sulphate, potassium chloride, potassium phosphate, sodium chloride, calcium nitrate, zinc chloride, ferric nitrate, sodium pyruvate, D-calcium pantothenate, tyrosine disodium salt, sodium selenite, zinc selenite.

7. Proteolytic Enzymes

Papain, collagenase (preferably type Ia, type II, type IV), serratiopeptidase, heparanase, DNAse, elastase, bromelain, bradykinase, Clostridium peptidase, enzymes expressed by Lactobacillus acidophilus, enzymes expressed by the genus Aspergillus, protease, aliinase, fibrinolysin.

8. Mucopolysaccharides

Hyaluronic acid, condroitin sulphates.

9. Sugars, Alcohols Derived Therefrom and Mixtures Thereof

Glucose, sucrose, glucans, mannans, glucomannans, fucose, fructose, heparan sulphates, pectins, starches, the alcohols derived therefrom.

10. Cell Culture Solutions

RPMI 1640 (is a basic medium for culturing mammalian and hybrid cell-lines), DMEM-LG (the culture medium DMEM is a modification of Eagle's minimal essential medium (EMEM) containing amino acids, salts, glucose, vitamins and iron; LG indicates that the glucose concentration is low), FBS (foetal bovine serum), F12 (cell culture solution containing a complete amino acid source), HANK'S solution (cell culture solution containing sodium bicarbonate), SyntheChol (NS0 Supplement, Sigma).

11. Haemoderivatives

Autologous serum prepared from the peripheral blood from tissue, cell and culture medium donors and recipients.

12. Parasympatholytic Substances

Adiphenine, aminocarbofluorene, anisotropine, anticholinesterases, atropine, benzatropine, cyclopentolate, clidinium, dicyclomine, dicycloverine, dioxyline, hexocyclium, ethaverine, glycopyrrolate, himbacine, ipratropium, mcn-a-343 (m-chlorophenyl-carbamol-oxybutinyl-trimethyl-amonium-chloride), methyl-scopolamine, metocramine, mepenzolate, metanteline, muscarine, omatropine, oxyphencyclimine, oxyphenonium, oxotremorine, piperidolate, poldine, pipenzolate, pirenzepine, pirenzepine analogue (AF-DX 116), pralidoxine, propanteline, propanteline bromide, prifinium, thiemonium, thiotropium, tolterodine, tripitramine, tropicamine, trospium, scopolamine; all the derivatives and the natural and synthetic alkaloids of the above listed substances. And preferably, according to IUPAC classification:

1) Anisotropine

[(8-methyl-8-azabicyclo[3.2.1]octo-3-yl)-2-propylpentanoate];

2) Anisotropine methylbromide;

3) Atropine

[(8-methyl-8-azabicyclo[3.2.1]octo-3-yl)3-hydroxy-2-phenyl-propanoate];

4) Atropine hydrochloride;

5) Atropine hyperduric;

6) Atropine methylbromide;

7) Atropine methylnitrate;

8) Atropine N-oxide;

9) Atropine sulphate;

10) Clidinium

[(4-methyl-4-azniabicyclo[2.2.2]octo-2-yl)-2-hydroxy-2,2-diphenyl-acetate];

11) Clidinium bromide;

12) Cyclopentolate

[2-dimethylaminoethyl-2-(1-hydroxycyclopentyl)-2-phenyl-acetate];

13) Cyclopentolate hydrochloride;

14) Dicyclomine

[2-diethylaminoethyl-1-cyclohexylcyclo-hexan-1-carboxylate];

15) Glycopyrrolate

[(1,1-dimethyl-2,3,4,5-tetrahydropyrrol-3-yl)-2-cyclopentyl-2-hydroxy-2-phenyl-acetate];

16) Isopropamide

[(3-carbamoyl-3,3-diphenyl-propyl)-methyl-dipropan-2-yl-ammonium];

17) Hexocyclium methylsulphate

[1-cyclohexyl-2(4,4-dimethyl-2,3,5,6tetrahydropyrazine-1-yl)-1-phenyl-ethanol; sulphonate-oxymethane];

18) Mepenzolate

[(1,1-dimethyl-3,4,5,6-tetrahydro-2H-pyridin-3-yl)2-hydroxy-2,2-diphenyl-acetate];

19) Methantheline

[diethyl-methyl-[2-(9H-xanthen-9-yl-carbonyl-oxy)-ethyl]ammonium];

20) Methylatropine

[(8,8-dimethyl-8-azoniabicyclo[3,2,1]octo-3-yl)3-hydroxy-2-phenyl-propanoate];

21) Methylatropine nitrate;

22) Homatropine;

[(8-methyl-8-azabicyclo[3.2.1]octo-3-yl)2-hydroxy-2-phenyl-acetate];

23) Homatropine hydrobromide;

24) Homatropine methylbromide;

25) Homatropine hydrochloride;

26) Oxyphencyclimine

[(1-methyl-5,6-dihydro-4H-pyrimidin-2-yl)methyl-2-cyclohexyl-2-hydroxy-2-phenyl-acetate];

27) Oxyphenonium [2-(2-cyclohexyl-2-hydroxy-2-phenyl-acetyl)oxyethyl-triethyl-ammonium];

28) Oxyphenonium bromide;

29) Pyrenzepine [5,11-Dihydro-11-[(4-methyl-1-piperazinyl)acetyl]-6H-pyridol[2,3b][1,4]benzodiazepin-6-one];

30) Propantheline [methyl-dipropan-2-yl-[2-(9H-xanthen-9-yl-carbonyloxy)ethyl ammonium];

31) Methyiscopolamine [7(S)-(1α,2β,4β,5α,7β)]-7-(3-hydroxy-1-oxo-2-phenyl-propoxy)-9,9-dimethyl-3-oxa-9-azoniatricyclo[3.3.1.0^{2,4}]nonan bromide];

32) Scopolamine [[7(S)-(1α,2β,4β,5α,7β)]-α-(hydroxymethyl)acid benzenacetic-9-methyl-3-oxa-9-azatricyclo[3.3.1.02,4]non-7-yl-ester];

33) Scopolamine hydrobromide;

34) Scopolamine hydrochloride;

35) Scopolamine methylbromide;

36) Scopolamine methylnitrate;

37) Scopolamine N-oxide;

38) Tifenamil

[1-(2-diethylaminoethylsulphanyl)-2,2-diphenyl-ethanone];

39) Tifenamil hydrochloride;

40) Tridihexethyl [(3-cyclohexyl-3-hydroxy-3-phenyl-propyl)-triethyl-ammonium];

41) Tridihexethyl chloride;

42) Tropicamide [N-ethyl-3-hydroxy-2-phenyl-N-(yridine-4-yl-methyl)propanamide];

43) Tropicamide hydrobromide;

44) Tropicamide hydrochloride.

Compositions

The compositions forming the subject of the present invention (hereinafter designated as PSL-BASE-1, PSL-BASE-2, PSL-BASE-FARMA, PSL-BASE-INFUS) for in vitro and in vivo use have been prepared using the above substances in the amounts indicated in tables 1 to 4.

TABLE 1
Composition of PSL-BASE-1

| Composition | per Litre | |
|---|---|---|
| Glucose | 1 | g |
| RPMI 1640 or DMEM-LG | as required to make 1 | |
| | litre of solution | |
| FCS | 50 | mL |
| F12 | 10 | mL |
| HANK'S solution | 20 | mL |
| MEM solution - non-essential | 20 | mL |
| amino acids | | |
| Autologous serum | 2 | mL |
| Selected growth factors | | |
| TGF-Beta3 | 20 | μg |
| LIF | 1,000,000 | units |
| IL-6 | 1,000,000 | units |
| Insulin | 10 | IU |
| Transferrin | 6.35 | mg |
| Zinc selenite | 3.18 | mg |
| Sodium selenite | 3.18 | mg |
| Linoleic acid | 10 | mg |
| PMA | 3 | nM |
| IL-2 | 1,000,000 | units |
| M-CSF | 50 | μg |
| HGF | 100 | μg |
| Ascorbic acid | 515 | mg |
| Insulin | 10 | units |
| Calcium gluconate | 0.1 | g |
| Dexamethasone 21-phosphate | 40 | μg |
| disodium salt | | |
| Parasympatholytics | per litre of solution | |
| Scopolamine | 200 | μg |

TABLE 2
Composition of PSL-BASE-2

| Composition | mg/L | |
|---|---|---|
| Methionine | 4.94 | |
| Cystine | 5.12 | |
| N-acetylcysteine | 1.00 | |
| Cysteine | 3.51 | |
| Glycine | 4.37 | |
| Leucine | 15.90 | |
| Proline | 4.67 | |
| Glutamine | 45.00 | |
| Biotin | 0.05 | |
| Pantothenic acid | 0.25 | |
| L-Ascorbic acid | 515.00 | |
| Retinol | 0.0015 | |
| Papain | 0.20 | |
| Hyaluronic acid | 0.50 | |
| Glucose | 2000.00 | |
| Supplemented solutions | L/sol | |
| RPMI 1640 or DMEM-LG | as required to make 1 | |
| | litre of solution | |
| FCS | 50 | mL |
| F12 | 10 | mL |
| HANK'S solution | 20 | mL |
| MEM solution - non-essential | 20 | mL |
| amino acids | | |
| Autologous serum | 2 | mL |
| | per Litre | |
| Selected growth factors | | |
| TGF-B3 | 20 | μg |
| LIF | 1,000,000 | units |
| IL-6 | 1,000,000 | units |
| Insulin | 10 | IU |
| Transferrin | 6.35 | mg |
| Zinc selenite | 3.18 | mg |
| Sodium selenite | 3.18 | mg |
| Linoleic acid | 10 | mg |
| PMA | 3 | nM |
| IL-2 | 1,000,000 | units |
| M-CSF | 50 | μg |
| HGF | 100 | μg |
| Calcium gluconate | 0.1 | g |
| Dexamethasone 21-phosphate | 40 | μg |
| disodium salt | | |
| Parasympatholytics | | |
| Scopolamine | 200 | μg |

TABLE 3
Composition of PSL-BASE-FARMA

| Composition | mg/L | |
|---|---|---|
| Methionine | 4.94 | |
| Cystine | 5.12 | |
| N-acetylcysteine | 1.00 | |
| Cysteine | 3.51 | |
| Glycine | 4.37 | |
| Leucine | 15.90 | |
| Proline | 4.67 | |
| Glutamine | 45.00 | |
| Biotin | 0.05 | |
| Pantothenic acid | 0.25 | |
| L-Ascorbic acid | 515.00 | |
| Retinol | 0.0015 | |
| Papain | 0.20 | |
| Hyaluronic acid | 0.50 | |
| Glucose | 2000.00 | |
| Transferrin | 6.35 | mg |
| Zinc selenite | 3.18 | mg |
| Sodium selenite | 3.18 | mg |
| Linoleic acid | 10 | mg |
| Calcium gluconate | 0.1 | g |
| Dexamethasone 21-phosphate | 40 | μg |
| disodium salt | | |
| Transferrin | 6.35 | mg |
| Glycerol base | as required to make | |
| | up 1 Kg | |
| Supplemented substances in | | |
| solution | L/sol | |
| Autologous serum | 2 | mL |
| Insulin | 10 | IU |
| Parasympatholytics | per Litre | |
| scopolamine | 200 | μg |

TABLE 4
Composition of PSL-BASE-INFUS

| Composition | mg/L | |
|---|---|---|
| Methionine | 4.94 | |
| Cystine | 5.12 | |
| N-acetylcysteine | 1.00 | |
| Cysteine | 3.51 | |
| Glycine | 4.37 | |
| Leucine | 15.90 | |
| Proline | 4.67 | |
| Glutamine | 45.00 | |
| Biotin | 0.05 | |
| Pantothenic acid | 0.25 | |
| L-Ascorbic acid | 515.00 | |
| Retinol | 0.0015 | |
| Papain | 0.20 | |
| Hyaluronic acid | 0.50 | |
| Glucose | 2000.0 | |
| Transferrin | 6.35 | mg |
| Zinc selenite | 3.18 | mg |
| Sodium selenite | 3.18 | mg |
| Linoleic acid | 10 | mg |
| Calcium gluconate | 0.1 | g |
| Dexamethasone 21-phosphate | 40 | μg |
| disodium salt | | |
| Supplemented solutions | L/sol | |
| Autologous serum | 2 | mL |
| Insulin | 10 | IU |
| Physiological solution | as required to make 1 | |
| (isotonic saline) | litre of final solution | |
| Parasympatholytics | per Litre | |
| Scopolamine | 200 | μg |

Cell Cultures

Isolation of Peripheral Blood Monocytes

Having withdrawn 40 mL of peripheral blood (six 7 mL EDTA tubes), the lympho-monocyte population is prepared as follows. The whole blood is split into two 20 mL aliquots and each aliquot is layered dropwise onto 25 mL of Ficoll-Paque (Amersham Pharmacia Biotech, Uppsala, Sweden) in 50 mL tubes (Lab-Tek, Nunc, Kamstrup, Denmark). The preparation is centrifuged once more at 1800 rpm for 25 minutes and the supernatant removed leaving 1 cm, and the transparent ring of monocytes which forms after the final centrifugation collected. The transparent ring thus obtained is diluted in 10 mL of RPMI 1640 (Life Technologies, Grand Island, N.Y., USA) and centrifuged at 1300 rpm for 10 minutes. The supernatant is then removed and the remainder washed a further twice by centrifugation at 1000 rpm for 10 minutes, and finally, the pellet is resuspended in 10 mL of RPMI 1640 with 10% FBS (Foetal Bovine Serum). The cells thus obtained are counted by means of a Burker chamber in order to obtain a final suspension of 5×10⁵ cells/mL. At this point the cells are seeded into Petri cells, 100 mm diameter plates (Falcon, Becton Dickinson, Labware Europe, Milan, Italy). The samples are incubated for 30 minutes at 37° C. with 5% CO₂. 30 minutes is the time required for the monocytes obtained to adhere to the plate, the cells are not allowed to stand any longer in order to avoid the lymphocytes also adhering. The cells have been washed, resuspended and grown as described in detail below.

Monocytoid Cell Line and Monocytes Isolated from Peripheral Blood

The cells have been washed 3 times by centrifugation at 160 g for 10 minutes at room temperature in RPMI 1640 medium (Life Technologies, Grand Island, N.Y., USA) and resuspended in 15 cm plates (Lab-Tek chamber slides, Nunc, Kamstrup, Denmark) at a final concentration of 1×10⁶ cells/mL in medium composed as follows:

100 units/mL penicillin

100 μg/mL streptomycin

160 mg/L gentamycin (Schering-Plough, Milan, Italy)

2 mM L-glutamine (Life Technologies; growth medium)

50 ng/mL M-CSF (macrophage colony stimulating factor, Peprotech Inc., NJ, USA)

1000 units/mL LIF (leukaemia inhibitory factor, Santa Cruz Biotechnology, CA, USA)

1000 units/mL IL-2 (human recombinant interleukin 2)

3 nM phorbol-12-myristate-13-acetate (PMA, (Santa Cruz Biotechnology, CA, USA).

Three types of controls have been prepared; one negative control (1) of untreated cells, one control (2) of cells treated with LIF (anti-leukaemic factor, leukaemia inhibitory factor) and one control (3) of cells treated with LIF (anti-leukaemic factor, leukaemia inhibitory factor), with M-CSF (macrophage colony stimulating factor, Peprotech Inc., NJ, USA), with PMA (3 nM phorbol-12-myristate-13-acetate, Santa Cruz Biotechnology, CA, USA) and with IL-2 (human recombinant interleukin 2) as described in detail hereinafter.

1. Control 1: The control cells not subjected to conditioning have been washed 3 times by centrifugation at 160 g for 10 minutes at room temperature in RPMI 1640 medium (Life Technologies, Grand Island, N.Y., USA) and resuspended in 15 cm plates (Lab-Tek chamber slides, Nunc, Kamstrup, Denmark) at a final concentration of 1×10⁶ cells/mL in RPMI 1640 medium supplemented with:

10% FCS (Celbio, Milan, Italy)

100 units/mL penicillin

100 μg/mL streptomycin

160 mg/L gentamycin (Schering-Plough, Milan, Italy)

2 mM L-glutamine (Life Technologies; growth medium).

2. Control 2: The cells have been washed 3 times by centrifugation at 160 g for 10 minutes at room temperature in RPMI 1640 medium (Life Technologies, Grand Island, N.Y., USA) and resuspended in 15 cm plates (Lab-Tek chamber slides, Nunc, Kamstrup, Denmark) at a final concentration of 1×10⁶ cells/mL in RPMI 1640 medium supplemented with:

10% FCS (Celbio, Milan, Italy)

100 units/mL penicillin

100 μg/mL streptomycin

160 mg/L gentamycin (Schering-Plough, Milan, Italy)

2 mM L-glutamine (Life Technologies; growth medium)

50 ng/mL M-CSF (macrophage colony stimulating factor, Peprotech. Inc., NJ, USA)

1000 units/mL LIF (leukaemia inhibitory factor, Santa Cruz Biotechnology, CA, USA)

1000 units/mL IL-2 (human recombinant interleukin 2)

3 nM phorbol-12-myristate-13-acetate (PMA, Santa Cruz Biotechnology, CA, USA).

All samples have been incubated for 15 days in a Heraeus thermostatically controlled incubator at a temperature of 37° C. in an atmosphere containing a constant flow of 5% CO₂ (v/v in air). It should be clarified that in all the samples, the medium has been regularly changed every 7 days, leaving 30% of the medium so as not to remove all the growth-essential cytokines produced by the cells.

Samples of all the cells in the study have been washed three times by centrifugation at 160 g for 10 minutes at 37° C. and subjected to cytofluorometric analysis (Epics Profile II, Coulter, Hialeath, Fla., USA) after labelling with the following anti-human mouse monoclonal antibodies (Mabs) conjugated to either R-phycoerythrin (PE) or Fluorescein-IsoThioCyanate (FITC): anti human CD14 (Santa Cruz Biotechnology, CA, USA), anti human CD34 (Santa Cruz Biotechnology, CA, USA), anti CD45 (Santa Cruz Biotechnology, CA, USA), anti c-Kit (Santa Cruz Biotechnology, CA, USA) and anti c-Met (Santa Cruz Biotechnology, CA, USA). When tested by cell cytofluorimetry (Facs), the cells are significantly positive for all the markers of stem cell expression (CD14, CD34, CD45, CD90, c-Kit, c-Met).

After the incubation period, the cells appear to be in a state of semi adhesion/suspension, with mixed oval and fibroblastoid morphologies. The cells, which have been harvested using 2% lidocaine (Sigma Aldrich, Milan, Italy) in PBS, have been washed 3 times by centrifugation at 160 g for 10 minutes at 37° C. in RPMI 1640 (Life Technologies, Grand Island, N.Y., USA) and have been incubated for a second time in accordance with the following description.

3. Control 3: the treated controls destined to remain undifferentiated pluripotent stem cella (e.g. PSC-THP1, ICLC PD No. 05005) have been resuspended at a final concentration of 1×10⁵ cells per mL in 6 well plates (Lab-Tek chamber slides, Nunc, Kamstrup, Denmark) in 0.5 mL per well of final solution composed of RPMI 1640 medium supplemented with:

10% FCS (Celbio, Milan, Italy)

100 units/mL penicillin,

100 μg/mL streptomycin

160 mg/L gentamycin (Schering-Plough, Milan, Italy)

2 mM L-glutamine (Life Technologies; growth medium)

50 ng/mL M-CSF (macrophage colony stimulating factor, Peprotech Inc., NJ, USA)

1000 units/mL LIF (leukaemia inhibitory factor, Santa Cruz Biotechnology, CA, USA)

1000 units/mL IL-2 (human recombinant interleukin 2)

3 nM phorbol-12-myristate-13-acetate (PMA, Santa Cruz Biotechnology, CA, USA).

4. Control 4: the monocytes and monocytoid cell controls, destined to be stimulated in the normal way towards hepatocytic specialisation, have been resuspended in 50 mL tubes (Lab-Tek, Nunc, Kamstrup, Denmark) at a final concentration of 2×10⁶ cells per mL in a final solution composed as follows:

100 units/mL penicillin

100 μg/mL streptomycin

160 mg/L gentamycin (Schering-Plough, Milan, Italy)

2 mM L-glutamine (Life Technologies; growth medium)

50 ng/mL M-CSF (macrophage colony stimulating factor, Peprotech Inc., NJ, USA)

1000 units/mL LIF (leukaemia inhibitory factor, Santa Cruz Biotechnology, CA, USA)

1000 units/mL IL-2 (human recombinant interleukin 2)

3 nM phorbol-12-myristate-13-acetate (PMA, (Santa Cruz Biotechnology, CA, USA)

100 ng/mL HGF (hepatocyte growth factdr, Peprotech Inc., NJ, USA)

5 μL/mL of non-essential amino acid solution (Sigma Aldrich, Milan, Italy).

5. Sample 1: the monocytes and monocytoid cell samples, destined for accelerated stimulation towards hepatocytic specialisation by the addition of parasympatholytic substances, have been resuspended in 50 mL tubes (Lab-Tek, Nunc, Kamstrup, Denmark) at a final concentration of 2×10⁶ cells per mL in a final solution composed as follows:

100 units/mL penicillin

100 μg/mL streptomycin

160 mg/L gentamycin (Schering-Plough, Milan, Italy)

2 mM L-glutamine (Life Technologies; growth medium)

50 ng/mL M-CSF (macrophage colony stimulating factor, Peprotech Inc., NJ, USA)

1000 units/mL LIF (leukaemia inhibitory factor, Santa Cruz Biotechnology, CA, USA)

1000 units/mL IL-2 (human recombinant interleukin 2)

3 nM phorbol-12-myristate-13-acetate. (PMA, (Santa Cruz Biotechnology, CA, USA)

100 ng/mL HGF (hepatocyte growth factor, Peprotech Inc., NJ, USA)

5 μL/mL of non-essential amino acid solution (Sigma Aldrich, Milan, Italy)

the addition of parasympatholytic substances:

0.2 μg/mL scopolamine (Buscopan®, Boehringer Ingelheim, Italy).

All samples and controls have been set-up in triplicate. Triplicate experiments, each consisting of four controls (1, 2, 3, 4) and one sample, have been incubated at 37° C. in an atmosphere with a constant flow of 5% CO₂ (v/v in air), for 30 days in total.

Isolation and Culture of Human Mesenchymal Pluripotent Stem Cells

Human mesenchymal pluripotent stem cells (MSCs, mesenchymal Stem Cells) have been extracted from samples of bone marrow (BM) taken from the femoral head (spongy tissue and bone marrow) during complete hip-replacement surgery. The nucleated cells have been separated by means of a density gradient (Ficoll-Paque, Amersham) and resuspended in low-glucose Dulbecco's modified Eagle's medium (DMEM-LG, Gibco, Grand Island, N.Y., USA) supplemented with 10% foetal bovine serum (FBS, Sigma), 10 U/mL penicillin G, and 40 μg/mL gentamycin. After 24 hours in culture, the medium, along with any cells in suspension, has been removed by aspiration and fresh medium added onto the adhered cells. The cells have been grown in 75 cm² flasks in an incubator with 5% CO₂ at a temperature of 37° C. When the cells reach 80% confluence, they are detached using trypsin-EDTA (5 minutes incubation at 37° C. followed by the addition of 5 mL of pure FBS, and the detached cells in suspension are harvested), washed twice by centrifugation at 160 g for 7 minutes at 37° C. in DMEM-LG medium (Gibco; Grand Island, N.Y., USA) free of any supplements. The cells in the pellets thus obtained are resuspended at a concentration of 1×10⁶ in fresh DMEM-LG medium (Gibco; Grand Island, N.Y., USA) supplemented with 10% foetal bovine serum (FBS, Sigma), 10 U/mL penicillin G, and 40 μg/mL gentamycin. The cells are reseeded after 1:4 dilution (final concentration 2.5×10⁴). In this study, cells after the third passage in continuous culture have been used.

Control 1: the controls (1) ‘destined to remain undifferentiated pluripotent stem cells have been grown for 15 days on 60 mm diameter plates (Falcon, Becton Dickinson, Labware Europe, Milan, Italy) in 10 mL of original culture medium (fresh DMEM-LG medium, Gibco; Grand Island, N.Y., USA) supplemented with 10% foetal bovine serum, Sigma, 10 U/mL penicillin G, and 40 μg/mL gentamycin.

Control 2: the samples destined to be stimulated in the normal way towards hepatocytic specialisation, detached and harvested as described above, have been resuspended in 50 mL tubes (Lab-Tek, Nunc, Kamstrup, Denmark) at a final concentration of 2×10⁶ cells per mL in a final solution composed as follows:

100 units/mL penicillin

100 μg/mL streptomycin

160 mg/L gentamycin (Schering-Plough, Milan, Italy)

2 mM L-glutamine (Life Technologies; growth medium)

50 ng/mL M-CSF (macrophage, colony stimulating factor, Peprotech Inc., NJ, USA)

1000 units/mL LIF (leukaemia inhibitory factor, Santa Cruz Biotechnology, CA, USA)

1000 units/mL IL-2 (human recombinant interleukin 2)

3 nM phorbol-12-myristate-13-acetate (PMA, (Santa Cruz Biotechnology, CA, USA)

100 ng/mL HGF (hepatocyte growth factor, Peprotech Inc., NJ, USA)

5 μL/mL of non-essential amino acid solution (Sigma Aldrich, Milan, Italy).

Sample 1: the samples destined for accelerated stimulation towards hepatocytic specialisation, detached and harvested as described above, have been resuspended in 50 mL tubes (Lab-Tek, Nunc, Kamstrup, Denmark) at a final concentration of 2×10⁶ cells per mL in a final solution composed as follows:

100 units/mL penicillin

100 μg/mL streptomycin

160 mg/L gentamycin (Schering-Plough, Milan, Italy)

2 mM L-glutamine (Life Technologies; growth medium)

50 ng/mL M-CSF (macrophage colony stimulating factor, Peprotech Inc., NJ, USA)

1000 units/mL LIF (leukaemia inhibitory factor, Santa Cruz Biotechnology, CA, USA)

1000 units/mL IL-2 (human recombinant interleukin 2)

3 nM phorbol-12-myristate-13-acetate (PMA, (Santa Cruz Biotechnology, CA, USA)

100 ng/mL HGF (hepatocyte growth factor, Peprotech Inc., NJ, USA)

5 μL/mL of non-essential amino acid solution (Sigma Aldrich, Milan, Italy)

the addition of parasympatholytic substances:

0.2 μg/mL scopolamine (Buscopan®, Boehringer Ingelheim, Italy).

All samples and controls have been set-up in triplicate. Triplicate experiments, each consisting of four controls (1, 2, 3, 4) and one sample, have been incubated at 37° C. in an atmosphere with a constant flow of 5% CO₂ (v/v in air), for 30 days.

Isolation and Culture of Synovial Cells

Synovial cells (SynCs) have been obtained from patients during exeresis and curettage of the synovial membrane inside the femoral-tibial joint cavity. Fresh synovial tissue sections have been disaggregated in collagenase (10 μg per mL of medium, Sigma) for two hours at 37° C. The pellet, washed twice in DMEM-LG (Gibco; Grand Island, N.Y., USA), free of any supplements, by centrifugation at 160 g for 10 minutes and then resuspended in complete low-glucose Dulbecco's modified Eagle's medium ([DMEM-LG] GIBCO; Grand Island, N.Y., USA) supplemented with 10% foetal bovine serum ([FBS] Sigma), 10 U/mL penicillin G, and 40 μg/mL gentamycin, is finally seeded into 75 cm² flasks and incubated in 5% CO₂ at a temperature of 37° C. After 24 hours, the medium, along with any cells still in suspension, has been aspirated off and fresh medium (DMEM-LG, Gibco; Grand Island, N.Y., USA, supplemented with 10% FBS, Sigma, 10 U/mL penicillin G, and 40 μg/mL gentamycin) has been added over the adhered cells. The cells have been grown in 75 cm² flasks in an incubator with 5% CO₂ at a temperature of 37° C. When the cells reach 80% confluence, they are detached using trypsin-EDTA (5 minutes incubation at 37° C. followed by the addition of 5 mL of pure FBS, and the detached cells in suspension are harvested), washed twice by centrifugation at 160 g for 7 minutes at 37° C. in DMEM-LG medium (Gibco; Grand Island, N.Y., USA) free of any supplements. The cells in the pellets thus obtained are resuspended at a concentration of 1×10⁶ in fresh DMEM-LG medium (Gibco; Grand Island, N.Y., USA) supplemented with 10% foetal bovine serum ([FBS] Sigma), 10 U/mL penicillin G, and 40 μg/mL gentamycin. The cells have been reseeded after 1:4 dilution (final concentration 2.5×10⁴). In this study, cells after the second passage in continuous culture have been used.

Sample 1: the samples destined to be stimulated towards hepatocytic specialisation, detached and harvested as described above, have been resuspended in 50 mL tubes (Lab-Tek, Nunc, Kamstrup, Denmark) at a final concentration of 2×10⁶ cells per mL in a final solution composed as follows:

100 units/mL penicillin

100 μg/mL streptomycin

160 mg/L gentamycin (Schering-Plough, Milan, Italy)

2 mM L-glutamine (Life Technologies; growth medium)

50 ng/mL M-CSF (macrophage colony stimulating factor, Peprotech Inc., NJ, USA)

1000 units/mL LIF (leukaemia inhibitory factor, Santa Cruz Biotechnology, CA, USA)

1000 units/mL IL-2 (human recombinant interleukin 2)

3 nM phorbol-12-myristate-13-acetate (PMA, (Santa Cruz Biotechnology, CA, USA)

100 ng/mL HGF (hepatocyte growth factor, Peprotech Inc., America, New Jersey)

5 μL/mL of non-essential amino acid solution (Sigma Aldrich, Milan, Italy).

Control 2: the samples destined to be stimulated in the normal way towards hepatocytic specialisation, detached and harvested as described above, have been resuspended in 50 mL tubes (Lab-Tek, Nunc, Kamstrup, Denmark) at a final concentration of 2×10⁶ cells per mL in a final solution composed as follows:

100 units/mL penicillin

100 μg/mL streptomycin

160 mg/L gentamycin (Schering-Plough, Milan, Italy)

2 mM L-glutamine (Life Technologies; growth medium)

50 ng/mL M-CSF (macrophage colony stimulating factor, Peprotech Inc., NJ, USA)

1000 units/mL LIF (leukaemia inhibitory factor, Santa Cruz Biotechnology, CA, USA)

1000 units/mL IL-2 (human recombinant interleukin 2)

3 nM phorbol-12-myristate-13-acetate (PMA, (Santa Cruz Biotechnology, CA, USA)

100 ng/mL HGF (hepatocyte growth factor, Peprotech Inc., NJ, USA)

5 μL/mL of non-essential amino acid solution (Sigma Aldrich, Milan, Italy).

Sample 1: the samples destined for accelerated stimulation towards hepatocytic specialisation, detached and harvested as described above, have been resuspended in 50 mL tubes (Lab-Tek, Nunc, Kamstrup, Denmark) at a final concentration of 2×10⁶ cells per mL in a final solution composed as follows:

100 units/mL penicillin

100 μg/mL streptomycin

160 mg/L gentamycin (Schering-Plough, Milan, Italy)

2 mM L-glutamine (Life Technologies; growth medium)

50 ng/mL M-CSF (macrophage colony stimulating factor, Peprotech Inc., NJ, USA)

1000 units/mL LIF (leukaemia inhibitory factor, Santa Cruz Biotechnology, CA, USA)

1000 units/mL IL-2 (human recombinant interleukin 2)

3 nM phorbol-12-myristate-13-acetate. (PMA, (Santa Cruz Biotechnology, CA, USA)

100 ng/mL HGF (hepatocyte growth factor, Peprotech Inc., NJ, USA)

5 μL/mL of non-essential amino acid solution (Sigma Aldrich, Milan, Italy)

the addition of parasympatholytic substances:

0.2 μg/mL scopolamine (Buscopan®, Boehringer Ingelheim, Italy).

All samples and controls have been set-up in triplicate. Triplicate experiments, each consisting of four controls (1, 2, 3, 4) and one sample, have been incubated at 37° C. in an atmosphere with a constant flow of 5% CO₂ (v/v in air), for 30 days in total.

Preliminary Cell Suspensions

During the entire experiment, including after stimulation towards assuming the hepatocytic phenotype as described above, cell samples have been obtained from all the study samples every 7 days of incubation.

All samples of the monocytoid cell lines and the semi-adhered peripheral blood monocytes have been tested every third day of incubation by immunofluorescence of samples adhering to cell culture slides.

Furthermore, samples of the same cells, taken every seventh day of incubation, have been incubated for 5-8 minutes with 2% lidocaine (Sigma Aldrich, Milan, Italy) in PBS and the solution thus obtained harvested, as described in the bibliography [1-3]. The cells have then been washed three times by centrifugation at 160 g for 10 minutes at 37° C. in RPMI 1640 (Life Technologies, Grand Island, N.Y., USA) without supplements. The cells obtained from the pellets of the samples and corresponding controls have been resuspended in 15 mL tubes (Lab-Tek chamber slides, Nunc, Kamstrup, Denmark) at a final concentration of 5×10⁵ cells/mL for subsequent phenotypic analysis (Western Blotting, direct immunofluorescence and FACS).

As described above in detail, every seventh day of incubation samples of all confluent MSC and SynC cells have been subjected to immunofluorescence testing of samples adhered onto sell culture slides.

Furthermore, every seventh day of incubation, samples of the same cells have been detached using trypsin-EDTA (5 minutes at a temperature of 37° C. followed by the addition of 5 mL of pure FBS and the detached cells in suspension harvested) and washed twice by centrifugation at 160 g for 7 minutes at 37° C. in DMEM-LG (Gibco; Grand Island, N.Y., USA) without any supplements. The cells obtained from the pellets of the samples and corresponding controls have been resuspended in 15 mL tubes (Lab-Tek chamber slides, Nunc, Kamstrup, Denmark) at a final concentration of 5×10⁵ cells/mL for subsequent phenotypic analysis (Western Blotting, direct immunofluorescence and FACS).

Western Blotting

Western blotting first makes use of denaturing electrophoresis (SDS-PAGE) in order to separate the various proteins according to molecular weight, by cancelling out the charges on the amino acids which might influence migration. The cell samples, suspended in lysis buffer (1% SDS, 30 mM Tris pH 6.8, 5% glycerol) to which protease inhibitors (Protease Inhibitor Cocktail, Calbiochem, San Diego, Calif., USA) have been added, have been incubated for 30 minutes at 4° C. The lysates thus obtained have been centrifuged at 12,000 rpm for 20 minutes at 4° C. and the supernatants collected; the protein concentrations of the samples have been measured using the Bio-Rad method (Benchmark Plus assay, Bio-Rad). Prior to electrophoresis, the samples have been boiled for 5 minutes in the presence of beta-mercaptoethanol and bromophenol blue. The samples have been subjected to electrophoresis on a 12% gel (SDS-PAGE) and then transferred onto a PVDF membrane (Perkin Elmer Inc.). The membranes have been saturated with methanol at room temperature and subsequently incubated overnight, at 4° C., with the following primary antibodies diluted 1:500 in PBS with 5% skimmed milk powder: anti-CD 34 (Santa Cruz Biotechnology, CA, USA), anti-CD 14 (Santa Cruz Biotechnology, CA, USA), anti-CD 45 (Santa Cruz Biotechnology, CA, USA), anti-CD 71 (Santa Cruz Biotechnology, CA, USA), anti-CD 90 (Santa Cruz Biotechnology, CA, USA), anti-CD 29 (Santa Cruz Biotechnology, CA, USA), anti-CD 105 (Santa Cruz Biotechnology, CA, USA), anti CD 117/c-KIT (Santa Cruz Biotechnology, CA, USA), anti c-MET (Santa Cruz Biotechnology, CA, USA), anti cytokeratin 7 (Santa Cruz Biotechnology, CA, USA), anti cytokeratin 8 (Santa Cruz Biotechnology, CA, USA), anti cytokeratin 18 (Santa Cruz Biotechnology, CA, USA), anti cytokeratin 19 (Santa Cruz Biotechnology, CA, USA), anti cytokeratin 7/17 (Santa Cruz Biotechnology, CA, USA), anti albumin (Rockland Immunochemicals, PA, USA), anti alpha-fetoprotein (Monosan Europa, Netherlands). After five washes, the membranes have been incubated with the corresponding secondary antibodies (1:1000) conjugated to horseradish peroxidase (HRP, SantaCruz Biotechnologies Inc., Santa Cruz, Calif., USA) for 1 hour at room temperature. The corresponding bands have been revealed using chemiluminescence liquid (Super Signalyestern Pico solution, Pierce Biotechnology Inc., Rockford, Ill., USA) and captured using photographic film.

Immunofluorescence Protocol

Cells in suspension have been incubated with 0.2 mM MitoTracker Red for 10 minutes at 37° C. After three washes by centrifugation at 160 g for 10 minutes at room temperature in PBS (pH 7.4), the cell pellets have been resuspended in a fixing solution of 4% paraformaldehyde in RPMI 1640 at pH 7.4, 1 hour at room temperature. After three washes in PBS, the cells have been resuspended in a solution of PBS and 0.1% Triton for 1 hour at 4° C. After three washes in PBS, the cells have been seeded onto slide covers and the liquid allowed to evaporate-off in air. The cells have been blocked with 20% normal goat serum for one hour, then incubated for 30 minutes with the following fluorescein isothiocyanate (FITC) conjugated anti-human monoclonal antibodies: anti-CD 34 (Santa Cruz Biotechnology, CA, USA), anti-CD 14 (Santa Cruz Biotechnology, CA, USA), anti-CD 45 (Santa Cruz Biotechnology, CA, USA), anti-CD 71 (Santa Cruz Biotechnology, CA, USA), anti-CD 90 (Santa Cruz Biotechnology, CA, USA), anti-CD 29 (Santa Cruz Biotechnology, CA, USA), anti-CD 105 (Santa Cruz Biotechnology, CA, USA), anti CD 117/c-KIT (Santa Cruz Biotechnology, CA, USA), anti c-KIT (Santa Cruz Biotechnology, CA, USA), anti c-MET (Santa Cruz Biotechnology, CA, USA), anti cytokeratin 7 (Santa Cruz Biotechnology, CA, USA), anti cytokeratin 8 (Santa Cruz Biotechnology, CA, USA), anti cytokeratin 18 (Santa Cruz Biotechnology, CA, USA), anti cytokeratin 19 (Santa Cruz Biotechnology, CA, USA), anti cytokeratin 7/17 (Santa Cruz Biotechnology, CA, USA), anti albumin (Rockland Immunochemicals, PA, USA), anti alpha-fetoprotein (Monosan Europa, Netherlands). Specific controls with the corresponding isotypes have been devised for each monoclonal antibody (Santa Cruz Biotechnology, CA, USA). The cover slips, mounted onto slides using moviol have been examined by light microscopy. The nuclei have been stained using Hoechst solution (dilution 1:1000). The cover slips, mounted onto slides using moviol have been examined by light microscopy [1-2].

Results

Light Microscopy: Incubation for 7 Days

After 7 days of incubation with RPMI 1640 medium, supplemented as described previously, and containing 1000 units/mL LIF, 50 ng/mL M-CSF and 3 nM phorbol-12-myristate-13-acetate (PMA) the treated controls displayed evidence of morphological transformation from rounded cells to an elongated fibroblastoid form adhering to the culture plate. The untreated controls only showed a rounded shape and were all in suspension.

Untreated peripheral blood monocytes (control 1) (RPMI 1640 medium, 10% FCS, 100 units/mL penicillin, 100 g/mL streptomycin, 160 mg/L gentamycin, 2 mM L-glutamine+PanLeuKine).

Peripheral blood monocytes (control 2) treated with anti-leukaemic factor (LIF, leukaemia inhibitory factor) exclusively (RPMI 1640 medium, 10% FCS, 100 units/mL penicillin, 100 g/mL streptomycin, 160 mg/L gentamycin, 2 mM L-glutamine, 1000 unit/mL LIF).

Peripheral blood monocytes (control 3) at 166 hours (day 7) of stimulation with M-CSF (RPMI 1640 Medium, supplemented as described previously, and containing 1000 units/mL LIF, 50 ng/mL M-CSF and 3 nM phorbol-12-myristate-13-acetate (PMA).

Untreated THP-1 cells (control 1) (RPMI 1640 medium, 10% FCS, 100 units/mL penicillin, 100 g/mL streptomycin, 160 mg/L gentamycin, 2 mM L-glutamine).

THP-1 cells (control 2) treated with anti-leukaemic factor (LIF, leukaemia inhibitory factor) exclusively (RPMI 1640 medium, 10% FCS, 100 units/mL penicillin, 100 g/mL streptomycin, 160 mg/L gentamycin, 2 mM L-glutamine, 1000 units/mL LIF).

THP-1 cells (control 3, PSC-THP-1) at 166 hours (day 7) of stimulation with M-CSF (RPMI 1640 medium, supplemented as described previously, and containing 1000 units/mL LIF, 50 ng/mL M-CSF and 3 nM phorbol-12-myristate-13-acetate (PMA)).

MSC cells (control 1) at 166 hours, day 7 of incubation (DMEM-LG, Gibco, Grand Island, N.Y., USA), supplemented with 10% foetal bovine serum, Sigma, 10 U/mL penicillin G, and 160 mg/L gentamycin).

Cells isolated from synovial fluid and membranes (control 1) at 166 hours, day 7 of incubation (DMEM-LG, Gibco, Grand Island, N.Y.; USA), supplemented with 10% foetal bovine serum, Sigma, 10 U/mL penicillin G, and 40 μg/mL gentamycin).

Light Microscopy: Incubation for 15 Days

The samples of peripheral blood monocytes and monocytoid cells (THP-1 cell line) after 15 days of incubation (seven with stem cell induction and seven with specific medium supplemented with differentiation-accelerating parasympatholytic substances) with RPMI 1640 medium, supplemented as described previously, and containing 1000 units/mL LIF, 50 ng/mL M-CSF, 100 ng/mL HGF and 3 nM phorbol-12-myristate-13-acetate (PMA) and 0.2 μg scopolamine showed morphological transformation of the rounded cells to a tetrahedral shape adhered to the culture plate, positive for the production of albumin, alpha-fetoprotein, glycogen, cytokeratins 7, 8, 17, 18 19, OV-6, c-MET receptor (FACS, immunohistochemistry, WB, PCR). The controls only showed rounded morphology and were all in suspension with negative results for albumin, alpha-fetoprotein, glycogen, cytokeratins 7, 8, 17, 18 and 19, OV-6.

Peripheral blood monocytes (control 4) at 344 hours (day 14 of overall incubation: 7 days of stem cell induction+7 days of induction of differentiation towards the hepatocytic phenotype), treated with specific medium (RPMI 1640, supplemented as described previously, and containing 1000 units/mL LIF, 50 ng/mL M-CSF and 3 nM phorbol-12-myristate-13-acetate (PMA) and with the addition of 5 μL/mL of non-essential amino acid solution and 100 ng/mL HGF) from day 7 to day 14 of incubation.

Peripheral blood monocytes (sample 1) at 344 hours (day 14 of overall incubation: 7 days of stem cell induction+7 days of induction of differentiation towards the hepatocytic phenotype), treated with specific medium (RPMI 1640, supplemented as described previously, and containing 1000 units/mL LIF, 50 ng/mL M-CSF and 3 nM phorbol-12-myristate-13-acetate (PMA) and with the addition of 5 μL/mL of non-essential amino acid solution and 100 ng/mL HGF+0.2 μg scopolamine) from day 7 to day 14 of incubation.

THP-1 cells (control 4) at 344 hours (day 14 of overall incubation: 7 days of stem cell induction 7 days of induction of differentiation towards the hepatocytic phenotype), treated with specific medium (RPMI 1640, supplemented as described previously, and containing 1000 units/mL LIF, 50 ng/mL M-CSF and 3 nM phorbol-12-myristate-13-acetate (PMA) and with the addition of 5 μL/mL of non-essential amino acid solution and 100 ng/mL HGF) from day 7 to day 14 of incubation.

THP1 cells (sample 1) at 344 hours (day 14 of overall incubation: 7 days of stem cell induction+7 days of induction of differentiation towards the hepatocytic phenotype), treated with specific medium (RPMI 1640, supplemented as described previously, and containing 1000 units/mL LIF, 50 ng/mL M-CSF and 3 nM phorbol-12-myristate-13-acetate (PMA) and with the addition of 5 μL/mL of non-essential amino acid solution and 100 ng/mL HGF+0.2 μg scopolamine) from day 7 to day 14 of incubation.

MSC cells (sample 1) at 344 hours (day 14 of overall incubation: 7 days of stem cell induction+7 days of induction of differentiation towards the hepatocytic phenotype), treated with specific medium (RPMI 1640, supplemented as described previously, and containing 1000 units/mL LIF, 50 ng/mL M-CSF and 3 nM phorbol-12-myristate-13-acetate (PMA) and with the addition of 5 μL/mL of non-essential amino acid solution and 100 ng/mL HGF+0.2 μg scopolamine) from day 7 to day 14 of incubation.

Cells isolated from synovial fluid and membranes (sample 1) at 344 hours (day 14 of overall incubation: 7 days of stem cell induction+7 days of induction of differentiation towards the hepatocytic phenotype), treated with specific medium (RPMI 1640, supplemented as described previously, and containing 1000 units/mL LIF, 50 ng/mL M-CSF and 3 nM phorbol-12-myristate-13-acetate (PMA) and with the addition of 5 μL/mL of non-essential amino acid solution and 100 ng/mL HGF+0.2 μg scopolamine) from day 7 to day 14 of incubation.

Immunofluorescence

The cells have been treated with anti-human monoclonal antibodies against albumin (Rockland Immunochemicals, PA, USA), conjugated to fluorescein isothiocyanate (FITC) for 30 minutes. Specific controls with the corresponding isotypes have been devised for each monoclonal antibody (Santa Cruz Biotechnology, CA, USA).

Peripheral blood monocytes (control 4) at 344 hours (day 14 of overall incubation: 7 days of stem cell induction+7 days of induction of differentiation towards the hepatocytic phenotype), treated with specific medium (RPMI 1640, supplemented as described previously, and containing 1000 units/mL LIF, 50 ng/mL M-CSF and 3 nM phorbol-12-myristate-13-acetate (PMA) and with the addition of 5 μL/mL of non-essential amino acid solution and 100 ng/mL HGF) from day 7 to day 14 of incubation. Labelling with anti-albumin FITC Mabs.

Peripheral blood monocytes (sample 1) at 344 hours (day 14 of overall incubation: 7 days of stem cell induction+7 days of induction of differentiation towards the hepatocytic phenotype), treated with specific medium (RPMI 1640, supplemented as described previously, and containing 1000 units/mL LIF, 50 ng/mL M-CSF and 3 nM phorbol-12-myristate-13-acetate (PMA) and with the addition of 5 μL/mL of non-essential amino acid solution and 100 ng/mL HGF+0.2 μg/mL scopolamine) from day 7 to day 14 of incubation. Labelling with anti-albumin FITC Mabs.

THP-1 cells (control 4) at 344 hours (day 14 of overall incubation: 7 days of stem cell induction+7 days of induction of differentiation towards the hepatocytic phenotype), treated with specific medium (RPMI 1640, supplemented as described previously, and containing 1000 units/mL LIF, 50 ng/mL M-CSF and 3 nM phorbol-12-myristate-13-acetate (PMA) and with the addition of 5 μL/mL of non-essential amino acid solution and 100 ng/mL HGF) from day 7 to day 14 of incubation. Labelling with anti-albumin FITC Mabs.

THP-1 cells (sample 1) at 344 hours (day 14 of overall incubation: 7 days of stem cell induction+7 days of induction of differentiation towards the hepatocytic phenotype), treated with specific medium (RPMI 1640, supplemented as described previously, and containing 1000 units/mL LIF, 50 ng/mL M-CSF and 3 nM phorbol-12-myristate-13-acetate (PMA) and with the addition of 5 μL/mL of non-essential amino acid solution and 100 ng/mL HGF+0.2 μg/mL scopolamine) from day 7 to day 14 of incubation. Labelling with anti-albumin FITC Mabs.

MSC cells (control 4) at 344 hours (day 14 of overall incubation: 7 days of stem cell induction+7 days of induction of differentiation towards the hepatocytic phenotype), treated with specific medium (RPMI 1640, supplemented as described previously, and containing 1000 units/mL LIF, 50 ng/mL M-CSF and 3 nM phorbol-12-myristate-13-acetate (PMA) and with the addition of 5 μL/mL of non-essential amino acid solution and 100 ng/mL HGF) from day 7 to day 14 of incubation. Labelling with anti-albumin FITC Mabs.

MSC cells (sample 1) at 344 hours (day 14 of overall incubation: 7 days of stem cell induction+7 days of induction of differentiation towards the hepatocytic phenotype), treated with specific medium (RPMI 1640, supplemented as described previously, and containing 1000 units/mL LIF, 50 ng/mL M-CSF and 3 nM phorbol-12-myristate-13-acetate (PMA) and with the addition of 5 μL/mL of non-essential amino acid solution and 100 ng/mL HGF+0.2 μg/mL scopolamine) from day 7 to day 14 of incubation. Labelling with anti-albumin FITC Mabs.

Cells isolated from synovial fluid and membranes (control 4) at 344 hours (day 14 of overall incubation: 7 days of stem cell induction+7 days of induction of differentiation towards the hepatocytic phenotype), treated with specific medium (RPMI 1640, supplemented as described previously, and containing 1000 units/mL LIF, 50 ng/mL M-CSF and 3 nM phorbol-12-myristate-13-acetate (PMA) and with the addition of 5 μL/mL of non-essential amino acid solution and 100 ng/mL HGF) from day 7 to day 14 of incubation. Labelling with anti-albumin FITC Mabs.

Cells isolated from synovial fluid and membranes (sample 1) at 344 hours (day 14 of overall incubation: 7 days of stem cell induction+7 days of induction of differentiation towards the hepatocytic phenotype), treated with specific medium (RPMI 1640, supplemented as described previously, and containing 1000 units/mL LIF, 50 ng/mL M-CSF and 3 nM phorbol-12-myristate-13-acetate (PMA) and with the addition of 5 μL/mL of non-essential amino acid solution and 100 ng/mL HGF+0.2 μg/mL scopolamine) from day 7 to day 14 of incubation. Labelling with anti-albumin FITC Mabs.

Western Blotting

The samples have been subjected to phenotypic analysis by Western blotting for the following markers: anti-CD 34 (Santa Cruz Biotechnology, CA, USA), anti-CD 14 (Santa Cruz Biotechnology, CA, USA), anti-CD 45 (Santa Cruz Biotechnology, CA, USA), anti-CD 71 (Santa Cruz Biotechnology, CA, USA), anti-CD 90 (Santa Cruz Biotechnology, CA, USA), anti-CD 29 (Santa Cruz Biotechnology, CA, USA), anti-CD 105 (Santa Cruz Biotechnology, CA, USA), anti CD 117/c-KIT (Santa Cruz Biotechnology, CA, USA), anti c-MET (Santa Cruz Biotechnology, CA, USA), anti cytokeratin 7 (Santa Cruz Biotechnology, CA, USA), anti cytokeratin 8 (Santa Cruz Biotechnology, CA, USA), anti cytokeratin 18 (Santa Cruz Biotechnology, CA, USA), anti cytokeratin 19 (Santa Cruz Biotechnology, CA, USA), anti cytokeratin 7/17 (Santa Cruz Biotechnology, CA, USA), anti albumin (Rockland Immunochemicals, PA, USA), anti alpha-fetoprotein (Monosan Europa, Netherlands). After five washes, the membranes have been incubated with the corresponding secondary antibodies (1:1000) conjugated to horseradish peroxidase (HRP, SantaCruz Biotechnologies Inc., Santa Cruz, CA, USA) for 1 hour at room temperature, as reported in the following tables.

Characterisation of Circulating Monocytes Versus Pluripotent and Differentiated Monocytic Stem Cells

The results pertaining to the expression of CD34, CD14, c-KIT and c-Met, cytokeratins 7, 8, 18, 19, 7/17, albumin and alpha-fetoprotein have been expressed as follows:

TABLE 5

| | | | | monocytes |
|---|---|---|---|---|
| | | | monocytes | Hepatocytic |
| Surface | | monocytes | Hepatocytic | type + |
| antigens | monocytes | PSCs | type | parasympath. |
| CD34 | +++ | ++++ | −−−−− | −−−−− |
| CD14 | ++ | +++ | + | + |
| c-KIT | ++ | +++ | + | + |
| c-Met | +++ | ++++ | +++ | ++ |
| Cytokeratin 7 | + | ++ | +++ | +++++ |
| Cytokeratin 8 | + | ++ | +++ | ++++ |
| Cytokeratin | ++ | +++ | +++ | +++ |
| 18 | | | | |
| Cytokeratin | ++ | +++ | +++ | +++++ |
| 19 | | | | |
| Cytokeratins | ++ | ++ | ++ | ++++ |
| 7/17 | | | | |
| Human | +/− | + | ++ | +++++ |
| albumin | | | | |
| alpha- | −−− | +/− | ++++ | ++ |
| fetoprotein | | | | |

−−−−− = absence of any fluorescence
+ = 1-5 fluorescent cells per optical field
++ = 6-10 fluorescent cells per optical field
+++ = 10-20 fluorescent cells per optical field
++++ = 20-50 fluorescent cells per optical field
+++++ > 50 fluorescent cells per optical field

Characterisation of THP-1 Versus PSCs-THP and PSC-THP1-like Liver Cells

The results pertaining to the expression of CD34, CD14, c-KIT, c-Met, cytokeratins 7, 8, 18, 19, 7/17, albumin and alpha-fetoprotein have been expressed as follows:

TABLE 6

| | | | | PSC-THP1 |
|---|---|---|---|---|
| | | | PSC-THP1 | Hepatocytic |
| | | PSC- | Hepatocytic | type + |
| Surface antigens | THP-1 | THP1 | type | parasympath. |
| CD34 | +++ | ++++ | +++ | −−−−− |
| CD14 | ++ | +++ | ++ | + |
| c-KIT | ++ | +++ | ++ | + |
| c-Met | +++ | ++++ | +++ | ++ |
| Cytokeratin 7 | + | ++ | ++ | +++++ |
| Cytokeratin 8 | + | ++ | ++ | ++++ |
| Cytokeratin 18 | ++ | +++ | ++ | +++ |
| Cytokeratin 19 | ++ | +++ | +++ | +++++ |
| Cytokeratins | ++ | ++ | +++ | ++++ |
| 7/17 | | | | |
| Human albumin | +/− | + | +++ | +++++ |
| alpha- | −−− | +/− | ++++ | ++ |
| fetoprotein | | | | |

−−−−− = absence of any fluorescence
+ = 1-5 fluorescent cells per optical field
++ = 6-10 fluorescent cells per optical field
+++ = 10-20 fluorescent cells per optical field
++++ = 20-50 fluorescent cells per optical field
+++++ > 50 fluorescent cells per optical field

Characterisation of the Control and Treated Mesenchymal Cells

The results pertaining to the expression of CD34, CD14, CD45, CD29, CD117/c-KIT, CD71, CD90, CD105, albumin and alpha-fetoprotein have been expressed as follows:

TABLE 7

| | | | Mesenchymal |
|---|---|---|---|
| | | Mesenchymal | cells |
| | | cells | Hepatocytic |
| Surface | Mesenchymal | Hepatocytic | type + |
| antigens | cells | type | parasympath. |
| CD34 | −−− | −−− | −−− |
| CD14 | −−− | −−− | −−− |
| CD45 | −−− | −−− | −−− |
| CD29 | ++ | ++ | + |
| c-KIT | +++ | ++ | + |
| CD71 | ++++ | +++ | ++ |
| CD90 | ++++ | +++ | + |
| CD105 | +++ | ++ | + |
| Human | +/− | + | ++ |
| albumin | | | |
| alpha- | −−− | ++ | + |
| fetoprotein | | | |

−−−−− = absence of any fluorescence
+ = 1-5 fluorescent cells per optical field
++ = 6-10 fluorescent cells per optical field
+++ = 10-20 fluorescent cells per optical field
++++ = 20-50 fluorescent cells per optical field
+++++ > 50 fluorescent cells per optical field

Characterisation of the Synovial Cells

The results pertaining to the expression of CD34, CD14, CD45, CD29, CD117/c-KIT, CD71, albumin and alpha-fetoprotein have been expressed as follows:

TABLE 8

| | | | Synovial |
|---|---|---|---|
| | | Synovial | cells |
| | | cells | Hepatocytic |
| Surface | Synovial | Hepatocytic | type + |
| antigens | cells | type | parasympath. |
| CD34 | −−− | −−− | −−− |
| CD14 | −−− | −−− | −−− |
| CD45 | −−− | −−− | −−− |
| CD29 | −− | −− | −− |
| c-KIT | +++ | ++ | + |
| CD71 | ++++ | +++ | ++ |
| Human albumin | +/− | + | +++ |
| alpha- | −−− | +++ | + |
| fetoprotein | | | |

−−−−− = absence of any fluorescence
+ = 1-5 fluorescent cells per optical field
++ = 6-10 fluorescent cells per optical field
+++ = 10-20 fluorescent cells per optical field
++++ = 20-50 fluorescent cells per optical field
+++++ > 50 fluorescent cells per optical field

### BIBLIOGRAPHY

1. Tsuchiya S, Yamabe M, Yamaguchi Y, Kobayashi Y, Konno T, Tada K. Establishment and characterization of a human acute monocytic leukaemia cell line (THP-1). Int. J. Cancer. 1980;26:171-6.2. Rabinovitch, M. & De Stefano, M. J. Cell shape changes induced by cationic anaesthetics. J. Exp. Med. 1976;143:290-304.

2. Rabinovitch, M. & De Stefano, M. J. Cell shape changes induced by cationic anaesthetics. J. Exp. Med. 1976;143:290-304.

3. Parker F. “Cute e ormoni/Skin and Hormones” in Williams R. H. eds: “Trattato di Endocrinologia/Endocrinology Discussion”. III° Italian Edition, Piccin, Padova. 1979; vol II°, chapter 23:1115-19.

1. A method for accelerating the differentiation of stem cells into cells with a tissue-specific phenotype, the method comprising
Providing the stem cells;
Providing at least one parasympatholytic substance and
Stimulating said stem cells with said at least one parasympatholytic substance.

2. A medicament for accelerating the differentiation of stem cells into cells with a tissue-specific phenotype, the medicament comprising at least one parasympatholytic substance as an active compound.

3. The medicament according to claim 2, wherein said parasympatholytic substance is selected from the group consisting of adiphenine, aminocarbofluorene, atropine, anisotropine, anticholinesterases, benzatropine, cyclopentolate, clidinium, dicyclomine, dicycloverine, dioxyline, hexocyclium, ethaverine, glycopyrrolate, himbacine, ipratropium, mcn-a-343 (m-chlorophenyl-carbamoloxybutinyl-trimethyl-ammonium-chloride), methyl-scopolamine, metocramine, mepenzolate, metanteline, muscarine, omatropine, oxyphencyclimine, oxyphenonium, oxotremorine, piperidolate, poldine, pipenzolate, pirenzepine, pirenzepine analogue (AFDX 116), pralidoxine, propanteline, propanteline bromide, prifinium, thiemonium, thiotropium, tolterodine, tripitramine, tropicamine, trospium, scopolamine, anisotropine methylbromide, atropine hydrochloride, atropine hyperduric, atropine methylbromide, atropine methylnitrate, atropine N-oxide, atropine sulphate, Clidinium bromide, Cyclopentolate hydrochloride, Isopropamide, Hexocyclium methylsulphate, Methantheline, Methylatropine, Methylatropine nitrate, Homatropine, Homatropine hydrobromide, Homatropine methylbromide, Homatropine hydrochloride, Oxyphenonium bromide, Propantheline, Methylscopolamine, Scopolamine, Scopolamine hydrobromide, Scopolamine hydrochloride, Scopolamine methylbromide, Scopolamine methylnitrate, Scopolamine N-oxide, Tifenamil, Tifenamil hydrochloride, Tridihexethyl, Tridihexethyl chloride, Tropicamide, Tropicamide hydrobromide, and Tropicamide hydrochloride.

4. The medicament according to claim 2, wherein said parasympatholytic substance is comprised in an amount ranging from 0.001 mg/L to 10 mg/L.

5. The medicament according to claim 2, wherein said parasympatholytic substance is comprised in an amount ranging from 0.02 mg/L to 1 mg/L.

6. The medicament according to claim 2, wherein said parasympatholytic substance is scopolamine, and wherein said scopolamine is comprised in an amount ranging from 0.01 mg/L to 0.4 mg/L.

7. The medicament according to claim 2, wherein said tissue-specific phenotype is selected from the group consisting of hepatocytic, chondrocytic, cardiomyocytic, endothelial, epithelial, osteocytic, haematopoietic, pancreatic, neuronal, glial, adipose, and myocytic.

8. A culture medium capable of accelerating the differentiation of stem cells into cells with a tissue specific phenotype , the culture medium comprising salts, amino acids, sugars, peptides, vitamins and/or vitamin factors required for growth of eukaryotic cells, the culture medium further comprising at least one parasympatholytic substance, provided that if the parasympatholytic substance is scopolamine, said parasympatholytic substance is present in an amount of from 0.02 mg/L to 1 mg/L.

9. The culture medium according to claim 8, the culture medium further comprising a growth factor or other factor capable of introducing differentiation into a tissue-specific phenotype.

10. The culture medium according to claim 9, wherein said growth factor or other factor capable of inducing differentiation into a tissue-specific phenotype is selected from the group consisting of TGF-Beta (transforming growth factor beta), LIF (leukaemia inhibitory factor), ITS (insulin-transferrin-selenium), insulin, HGF (hepatocyte growth factor), M-CSF (macrophage colony stimulating factor), dexamethasone 21-phosphate disodium salt, calcium gluconate, retinoic acid, retinol, linoleic acid, and autologous serum.

11. The culture medium according to claim 8, wherein said parasympatholytic substance is selected from the group consisting of adiphenine, aminocarbofluorene, atropine, anisotropine, anticholinesterases, benzatropine, cyclopentolate, clidinium, dicyclomine, dicycloverine, dioxyline, hexocyclium, ethaverine, glycopyrrolate, himbacine, ipratropium, mcn-a-343 (m-chlorophenyl-carbamoloxybutinyl-trimethyl-ammonium-chloride), methyl-scopolamine, metocramine, mepenzolate, metanteline, muscarine, omatropine, oxyphencyclimine, oxyphenonium, oxotremorine, piperidolate, poldine, pipenzolate, pirenzepine, pirenzepine analogue (AFDX 116), pralidoxine, propanteline, propanteline bromide, prifinium, thiemonium, thiotropium, tolterodine, tripitramine, tropicamine, trospium, scopolamine, anisotropine methylbromide, atropine hydrochloride, atropine hyperduric, atropine methylbromide, atropine methylnitrate, atropine N-oxide, atropine sulphate, Clidinium bromide, Cyclopentolate hydrochloride, Isopropamide, Hexocyclium methylsulphate, Methantheline, Methylatropine, Methylatropine nitrate, Homatropine, Homatropine hydrobromide, Homatropine methylbromide, Homatropine hydrochloride, Oxyphenonium bromide, propantheline, Methylscopolamine, Scopolamine, Scopolamine hydrobromide, Scopolamine hydrochloride, Scopolamine methylbromide, Scopolamine methylnitrate, Scopolamine N-oxide, Tifenamil, Tifenamil hydrochloride, Tridihexethyl, Tridihexethyl chloride, Tropicamide, Tropicamide hydrobromide, and Tropicamide hydrochloride.

12. The culture medium according to claim 8, wherein the culture medium comprises the parasympatholytic substance in an amount ranging from 0.001 mg/L to 10 mg/L, provided that the parasympatholytic substance is not scopolamine.

13. The culture medium according to claim 8, wherein the culture medium comprises the parasympatholytic substance in an amount ranging from 0.02 mg/L to 1 mg/L.

14. The culture medium according to claim 8, wherein the culture medium comprises the parasympatholytic substance scopolamine in an amount ranging from 0.01 mg/L to 0.4 mg/L.

15. The culture medium according to claim 8, wherein said tissue-specific phenotype is selected from the group consisting of hepatocytic, chondrocytic, cardiomyocytic, endothelial, epithelial, osteocytic, haematopoietic, pancreatic, neuronal, glial, adipose, and myocytic salts.

16. A method for accelerating the differentiation of stem cells into cells having a tissue-specific phenotype, the method comprising the steps of:
providing the stem cells;
providing a culture medium comprising salts, amino acids, sugars, peptides, vitamins and/or vitamin factors required for growth of eukaryotic cells, the culture medium further comprising at least one parasympatholytic substance and at least one growth factor or other factor capable of inducing differentiation into a tissue-specific phenotype;
growing said stem cells in said culture medium to obtain cells with a tissue-specific phenotype.

17. The method according to claim 16, wherein said culture medium further comprises at least one of a macrophage colony stimulating factor and a leukaemia inhibitory factor.

18. The method according to claim 16 or 17, wherein said culture medium further comprises at least one interleukin.

19. The method according to claim 16, wherein said culture medium further comprises at least one antibiotic.

20. The method according to claim 16, wherein said culture medium further comprises phorbol-12-myristate13-acetate.

21. The method according to claim 16, wherein said other factor capable of inducing differentiation into a tissue-specific phenotype is selected from the group consisting of TGF-Beta (transforming growth factor beta), LIF (leukaemia inhibitory factor), ITS (insulin-transferrin-selenium), insulin, HGF (hepatocyte growth factor), M-CSF (macrophage colony stimulating factor), dexamethasone 21-phosphate disodium salt, calcium gluconate, retinoic acid, retinol, linoleic acid, and autologous serum.

22. A method for accelerating the differentiation of stem cells into cells with a tissue-specific phenotype in a patient in need thereof, the method comprising administering to said patient the medicament according to claim 2.

23. The culture medium of claim 8, wherein the eukaryotic cells are mammalian cells or human cells.

24. The culture medium according to claim 15, wherein said culture medium further comprises a vitamin medium the vitamin medium comprising peptides and vitamins.

25. The method of claim 16, wherein said eukaryotic cells are selected from the group consisting of mammalian cells and human cells.

26. The method of claim 16, wherein providing a culture medium is performed by
providing a culture medium comprising salts, amino acids, sugars, peptides, vitamins and/or vitamin factors required for growth of eukaryotic cells, the culture medium further comprising at least one parasympatholytic substance; and
supplementing said culture medium with at least one growth factor or other factor capable of inducing differentiation into a tissue-specific phenotype.

27. The method of claim 18, wherein said at least one interleukin is selected from the group consisting of interleukin-2 and interleukin-6.

28. The method of claim 19, wherein said at least one antibiotic is selected from the group consisting of gentamycin, penicillin and streptomycin.
